(19) United States
(12) Patent Application Publication
(10) Pub. No.: US 2019/0313184 A1
(43) Pub. Date: Oct. 10, 2019

(54) Headphone with transdermal electrical nerve stimulation

(71) Applicant: Richard Michael Truhill, Plano, TX (US)

(72) Inventor: Richard Michael Truhill, Plano, TX (US)

(21) Appl. No.: 15/932,660

(22) Filed: Apr. 5, 2018

(51) Int. Cl. H04R 1/46; A61M 21/02

(52) U.S. Cl. CPC H04R 1/46; A61M 2021/0027; A61M 2205/8206; A61M 21/02

(57) ABSTRACT

An electronic wearable apparatus for providing bimodal stimulation to auditory system and somatosensory system of a user is disclosed herein. The apparatus includes a head-mounted wearable housing that is attached to an auditory system and a somatosensory system of the user. The apparatus also includes a wearable audio output structure configured to apply audio stimulation to the auditory system of the user. The apparatus further includes a wearable transdermal electrical transducer configured to apply a somatosensory stimulation signal to the somatosensory system of the user through a transdermal electrical contact which is in direct contact with a portion of surface structure of the user for stimulating the somatosensory system. The apparatus further includes an electronic control processor that is connected to the audio output structure and the transdermal electrical transducer and configured to synchronize the bimodal stimulations to create a nerve stimulation to improve user's body.

### FIELD

The present disclosure relates to a device providing bimodal stimulation to auditory and somatosensory systems of a user, utilizing headphone speakers to provide auditory stimulation while transdermal electrical contacts deliver electrical impulses for somatosensory stimulation. Although the application of the present disclosure is focused on tinnitus for clarity and brevity, applications of the proposed disclosure cover a wide range of other problems, including pain, depression, and epilepsy, which have been well researched, and could possibly be applicable to other problems based on future research.

### BACKGROUND

Neuroplasticity, also known as brain plasticity and neural plasticity, is the brain's ability to reorganize itself by forming new neural connections, allowing neurons (nerve cells) to adjust their activities in response to new situations or to changes in their environment. Such reorganization has been shown to take place in response to bimodal stimulation of the auditory and somatosensory systems, resulting in improved objective and subjective measures of tinnitus.

Transdermal electrical nerve stimulation (TENS) has been used successfully in many applications including treatment of pain, depression, epilepsy, and tinnitus. Specifically for reducing tinnitus, it has been shown that stimulation of the vagus nerve combined with auditory stimulation provides the best results. Using this information, methods and devices have been developed to reduce tinnitus; however, some applications require surgical implantation of a subcutaneous device that provides electrical stimulation of the vagus nerve. Non-surgical options are available; however, they require the use of adhesively attached electrodes. Convenient non-clinical uses, such as in the home, have not been adequately addressed. In the treatment of tinnitus, a comfortable, easy-to-use non-surgically-implanted neurostimulator device is needed to provide more convenient treatment options.

Early research on bimodal pairing of auditory stimulation and somatosensory stimulation to reduce tinnitus used a surgical procedure in which a small transcutaneous electrical stimulation (TES) device was implanted in the user and connected to a wire which was wrapped around the vagus nerve. The implanted device, which contained its own power supply, received wireless control signals and sent electrical stimulation to the vagus nerve. The somatosensory stimulation was accompanied by auditory stimulation using sound output via headphones, either simultaneously with the somatosensory stimulation or at various combinations of timing, frequencies, power levels, etc. Whereas early transcutaneous devices required an invasive procedure such as surgery, later devices replaced the implanted device with an external transdermal electrical nerve stimulation (TENS) control device which was designed to be utilized with electrodes adhesively applied to the intact skin surface of the user.

The proposed disclosure allows a user to combine auditory stimulation, using one or a pair of headphones, with somatosensory stimulation, using TENS, without having to undergo surgery or using adhesively applied electrodes. The apparatus can house only the TENS contact electrodes, or it can also house the TENS control unit as well. It can be externally powered or powered by one or more internal power sources such as a battery. It can receive control inputs and/or power inputs by means of wires or wirelessly.

Using the disclosure proposed herein, combined with appropriate external hardware and software, users are able to achieve reduction in tinnitus more conveniently and effectively. The disclosed disclosure allows each user to choose an embodiment of the device they feel provides the most convenient application for them.

The proposed disclosure can be used with TENS; it can be configured as ordinary headphones; or it can be configured as a headset with a built-in microphone.

The prior art teaches various bimodal stimulation devices. Among these are:

U.S. Pat. No. 9,682,232 to Susan Shore, David Martel and Seth Koehler teaches a device for treating tinnitus utilizing a bimodal stimulation device configured to generate an audible stimulation signal while generating a somatosensory stimulation signal, either simultaneously or in a timed manner, to stimulate a somatosensory system of a subject (i.e., a user) utilizing various timing and duration patterns resulting in reduction in objective or subjective measures of tinnitus. A wearable transdermal neurostimulator that is connectable to a subject with an electrode apparatus is therein claimed. The Shore et al. Description states: “In some examples, the probes are deep brain region probes that stimulate the somatosensory system. Thus in some examples, the somatosensory stimulation signal to a subject results from applying stimulation to a surface region of the brain of the subject or to a surface structure on the face or a surface structure on the neck of the subject to stimulate the somatosensory system. These stimulations, whether auditory stimulation and somatosensory stimulation, may be provided through a mechanical or electrical stimulation.”

U.S. Pat. No. 9,393,401 to Isy Goldwasser, Sumon K. Pal, Jonathan Charlesworth, Wing Law, Jay Frederick Hamlin, Daniel Z. Wetmore, William J. Tyler, and Douglas Jeffery teaches wearable neurostimulator apparatuses connected to an electrode assembly that is adapted to be attached to the user's body.

U.S. Pat. No. 9,002,458 to Sumon K. Pal, Jonathan Charlesworth, Remi Demers, Daniel Z. Wetmore, Isy Goldwasser, William J. Tyler, Raymond L. Gradwohl, Phillip Lamb, and Christopher Voss describes apparatuses and methods for transdermal electrical stimulation, including transcranial electrical stimulation, to induce neuromodulation utilizing electrodes attached to the head or neck with adhesive.

None of these prior art references describe the present disclosure.

### SUMMARY

To overcome the problems stated above, the present disclosure provides a means for bimodal stimulation with simultaneous stimulation of auditory and somatosensory systems of a user, utilizing headphone speakers to provide auditory stimulation while transdermal electrical contacts deliver electrical impulses to stimulate the vagus nerves. Although usually referred to in the singular, there are actually both right and left vagus nerves that descend from the brain along the right and left sides of the neck.

An apparatus disclosed herein consists of a bimodal pairing control module, which then controls both auditory stimulation and somatosensory stimulation. Control inputs and/or power inputs are sent to the disclosed disclosure. The auditory control inputs utilize internal or external power sources to produce sound in audio output structure (or component), such as headphone speakers or bone conduction sound hearing structure, while the somatosensory control inputs utilize internal or external power sources to produce electrical current through the skin of the user with transdermal electrical contacts directly or through electrodes, which can be fixed or replaceable.

In one embodiment, an electronic wearable apparatus for providing bimodal stimulation to auditory system and somatosensory system of a user is disclosed herein. The apparatus includes a head-mounted wearable housing that is attached to an auditory system and a somatosensory system of the user. The apparatus also includes a wearable audio output structure configured to apply audio stimulation to the auditory system of the user. The apparatus further includes a wearable transdermal electrical transducer configured to apply a somatosensory stimulation signal to the somatosensory system of the user through a transdermal electrical contact which is in direct contact with a portion of surface structure of the user for stimulating the somatosensory system. The apparatus further includes an electronic control processor that is connected to the audio output structure and the transdermal electrical transducer and configured to synchronize the bimodal stimulations to create a nerve stimulation to improve user's body.

In another embodiment, a method for providing bimodal stimulation to auditory system and somatosensory system of a user is disclosed. The method includes providing the user a head-mounted wearable housing with electronic input and output. The method also includes providing the user a wearable audio output structure having tunable frequency and signal tracking capability that creates an audio stimulation for the user. The method further includes providing the user a wearable transdermal electrical transducer, and the transducer is configured to apply a somatosensory stimulation signal to the somatosensory system of the user through a transdermal electrical contact that is in direct contact with a portion of a surface structure of the user for stimulating the somatosensory system. The method further having an electronic control processor to control the audio output structure and the transdermal electrical transducer, and synchronize the bimodal stimulations to create a nerve stimulation to improve user's body states.

In another embodiment, an apparatus for providing bimodal stimulation to an auditory system and a somatosensory system of a user is provided. The apparatus includes a housing configured to hold an audio output component and a transdermal electrical component. The audio output component is configured to create an audio stimulation for the user and the transdermal electrical component is configured to apply a somatosensory stimulation signal to the somatosensory system of the user through a transdermal electrical contact that is in direct contact with a portion of a surface structure of the user for stimulating the somatosensory system.

In one embodiment, a pair of audio output structure (or component) is attached to a housing designed to be worn on the head of the user as with any conventional headphones. Said audio output structure (or component), generally referred to herein as “headphones” or “speakers”, may be of any design such as ear cushions with speakers, ear buds, earphones, etc.; and they may be air-conducting, bone-conducting, or any combination thereof.

In another embodiment the disclosure is configured for use with behind the neck type of headphones.

Attached to said headphones or said housing are transdermal electrical contacts configured to make contact with a body part of the user, specifically the neck. The headphone speakers provide auditory stimulation, such as a frequency tone, a combination of tones, or music; and the transdermal electrical contacts provide somatosensory stimulation to the nervous system of the user, specifically to the vagus nerves.

Said transdermal electrical contacts can be fixed, or they can be moveable, as by sliding or rotating, etc., so that when said contacts are stored or retracted, the apparatus can be used as conventional headphones without somatosensory stimulation.

In one embodiment said audio output structure (or component) and said transdermal electrical contacts can receive control signals and/or power inputs via wires. In another embodiment they can receive wireless signals and/or inputs; or inputs can be any combination of wired and wireless.

Positive contact between said transdermal electrical contacts and the user's body part can be maintained by the spring tension of said housing or with the aid of auxiliary spring tensioning devices. Said spring tensioning devices can be used to exert pressure against said transdermal electrical contacts or against said electrodes.

The apparatus can be configured as headphones or as a headset with the addition of some type of microphone.

The apparatus could be powered remotely, as with wired inputs, or utilize internal power source(s), such as a battery.

Said housing could contain other components which would not affect the proposed disclosure. Thus, the disclosure can be adapted to a wide range of designs.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a block diagram showing an exemplary apparatus for providing bimodal stimulation in accordance with some embodiments.

FIG. 2 is a perspective diagram of one embodiment of the present disclosure wherein transdermal electrical contacts are fixed.

FIG. 3 is a perspective diagram of a second embodiment of the present disclosure wherein transdermal electrical contacts are moveable, and in this configuration they are stored so the disclosure can be used as conventional headphones without somatosensory stimulation.

FIG. 4A is a perspective diagram of a transdermal electrical contact configuration wherein said contacts can slide into place for somatosensory stimulation, or they can be stored for use as conventional headphones without somatosensory stimulation.

FIG. 4B is a perspective diagram of a transdermal electrical contact configuration wherein said contacts can be rotated into place for somatosensory stimulation, or they can be stored for use as conventional headphones without somatosensory stimulation.

FIG. 4C is a perspective diagram of a transdermal electrical contact configuration wherein spring tension is used to maintain positive contact of said electrodes with a user's body part. Also shown is a replaceable electrode design with a large contact area.

FIG. 5 is a perspective diagram of an embodiment of the present disclosure as a headset with a microphone.

FIG. 6 is a perspective view of the disclosure configured for wired inputs.

FIG. 7 is a perspective view of the disclosure configured for wireless inputs.

FIG. 8 illustrates the disclosure as worn by a user in accordance with an exemplary embodiment.

FIG. 9 is a perspective diagram of an embodiment of the disclosure configured with a behind the neck type of headset.

### DETAILED DESCRIPTION OF THE DISCLOSURE

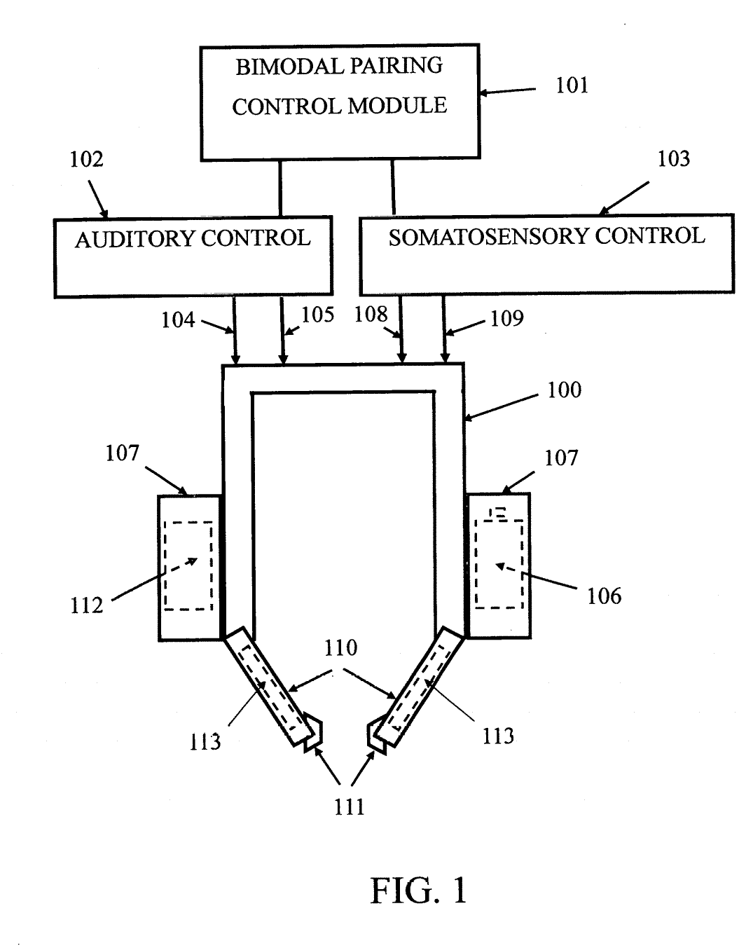

FIG. 1 is a block diagram showing an exemplary apparatus for providing bimodal stimulation. Bimodal pairing control module 101 coordinates auditory stimulation and somatosensory stimulation in conjunction with an electronic control process which includes a auditory control unit 102 and a somatosensory control unit 103. Auditory control inputs 104 to the disclosure 100 utilize external power source 105 or internal power source 106 to produce sound in audio output structure (or component) 107. The somatosensory control unit can be external 103 or internal 112. In the case of an external somatosensory control unit 103, somatosensory control inputs 108 to the disclosure 100 utilize external power source 109 or internal power source 106 to produce electrical current to the skin (not shown) of the user through transdermal electrical contacts 110 of a transdermal electrical transducer (or component) 113 or through electrodes 111, which can be fixed or replaceable. In the case of an internal somatosensory control unit 112, the function would be the same, but the somatosensory control inputs would be internal.

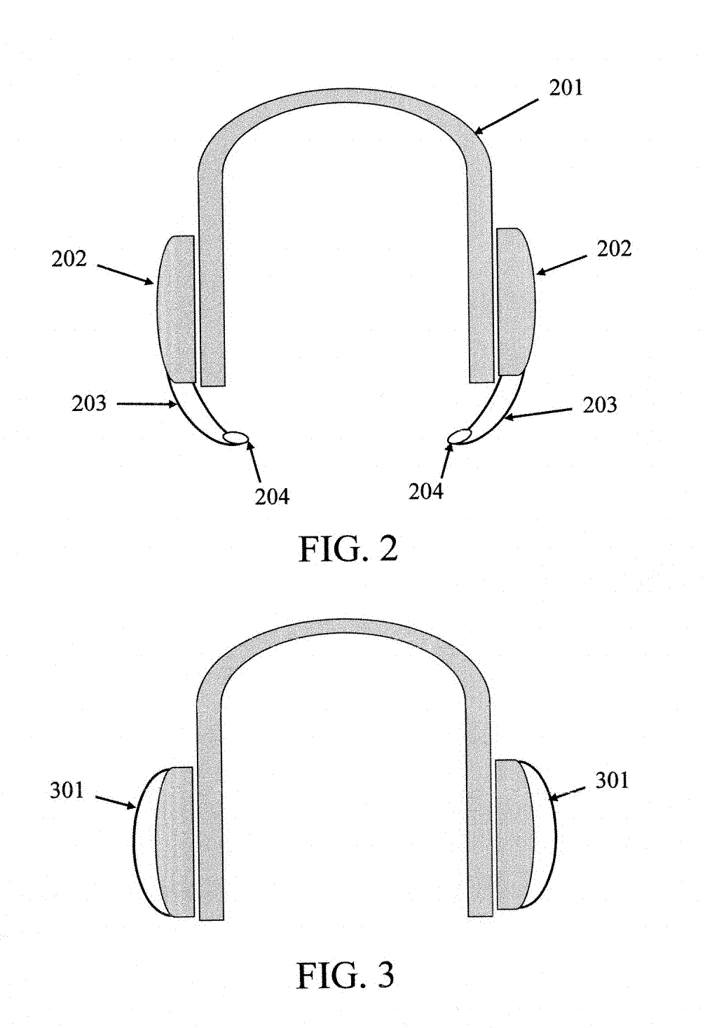

FIG. 2 is a perspective diagram of one embodiment of the present disclosure, including a housing 201, headphones 202, transdermal electrical contacts 203, and electrodes 204. Headphones 202 house audio output structure (or component) which provides auditory stimulation to a user, and transdermal electrical contacts 203 provide somatosensory stimulation by delivering electrical impulses to the skin of a user either directly or through electrodes 204. Electrodes 204 can be configured as integral with transdermal electrical contacts 203, or they can be removable and replaceable.

FIG. 3 is a perspective diagram of a second embodiment of the present disclosure wherein transdermal electrical contacts 301 are moveable; and in this configuration they are shown to be stored so the disclosure can be used as conventional headphones without somatosensory stimulation.

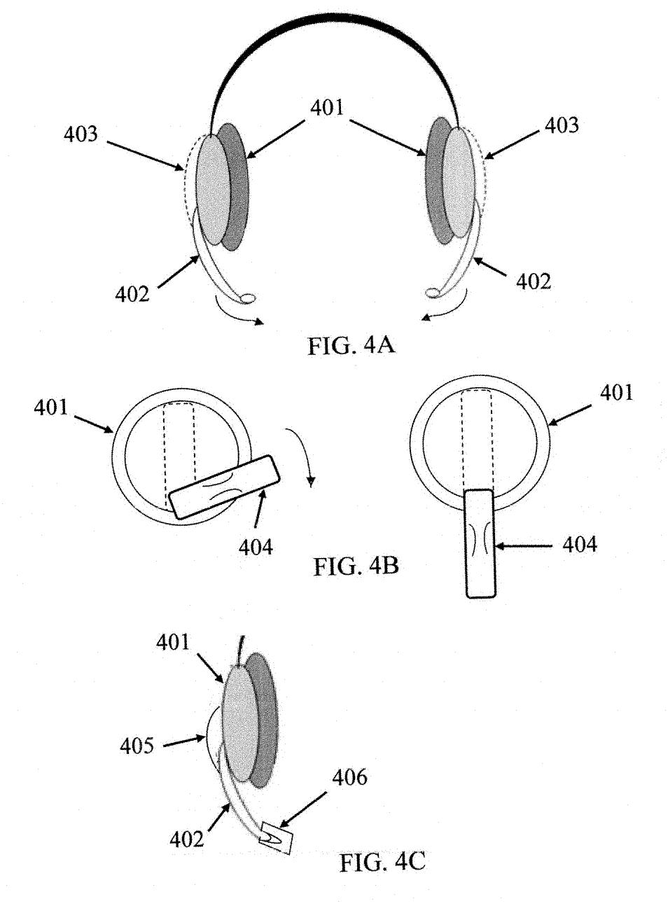

FIG. 4A is a perspective diagram of a transdermal electrical contact configuration, wherein a user can slide transdermal electrical contacts 402 into place for somatosensory stimulation as shown; or said contacts 402 can be stored as in position 403, so the apparatus can be used as conventional headphones without somatosensory stimulation. Moveable contacts 402 are shown in this illustration to be in position to deliver electrical impulses to a user's body part (not shown). In this embodiment transdermal electrical contacts can be retracted to position 403, shown by a dashed line, if desired.

FIG. 4B is a perspective diagram of a transdermal electrical contact embodiment wherein transdermal electrical contact 404 can be rotated into place for somatosensory stimulation, or said contact 404 can be stored so the apparatus can be used as conventional headphones without somatosensory stimulation. Moveable contact 404 is shown as it is rotated into position to deliver electrical impulses to a user's body part (not shown). In this embodiment transdermal electrical contact 404 can be rotated to a stored position, shown by dashed lines, if desired.

FIG. 4C is a perspective diagram of a transdermal electrical contact configuration wherein spring tension is used to maintain positive contact of electrode 406 with a user's body part (not shown). Spring 405 exerts pressure against transdermal electrical contact 402, bearing against another part of the apparatus (in this case headphone 401), causing large area electrode 406 to remain firmly in contact with a user's body part (not shown) at all times.

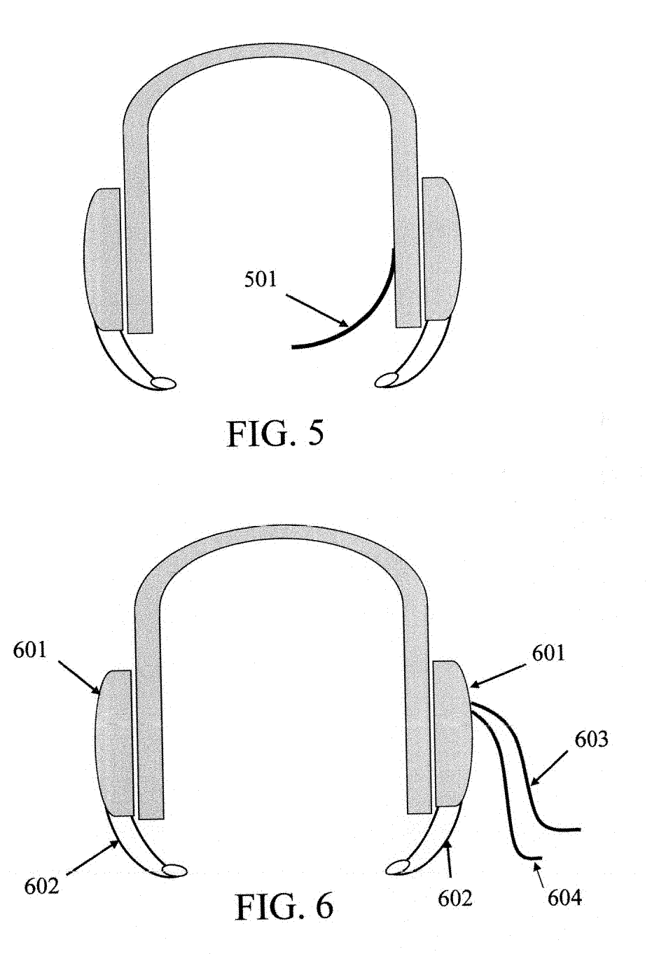

FIG. 5 is a perspective diagram of an embodiment of the present disclosure including microphone 501; thus, the apparatus can be used as a headset in addition to providing auditory stimulation and somatosensory stimulation.

FIG. 6 is a perspective view of the disclosure configured for wired inputs. Wire 603 provides a means for control inputs and/or power inputs to headphone speakers 601. Wire 604 provides a means for control inputs and/or power inputs to transdermal electrical contacts 602. Wires 603 and 604 are shown respectively as single lines, but said wires 603 and 604 can in fact be any number of separately insulated wires or cables.

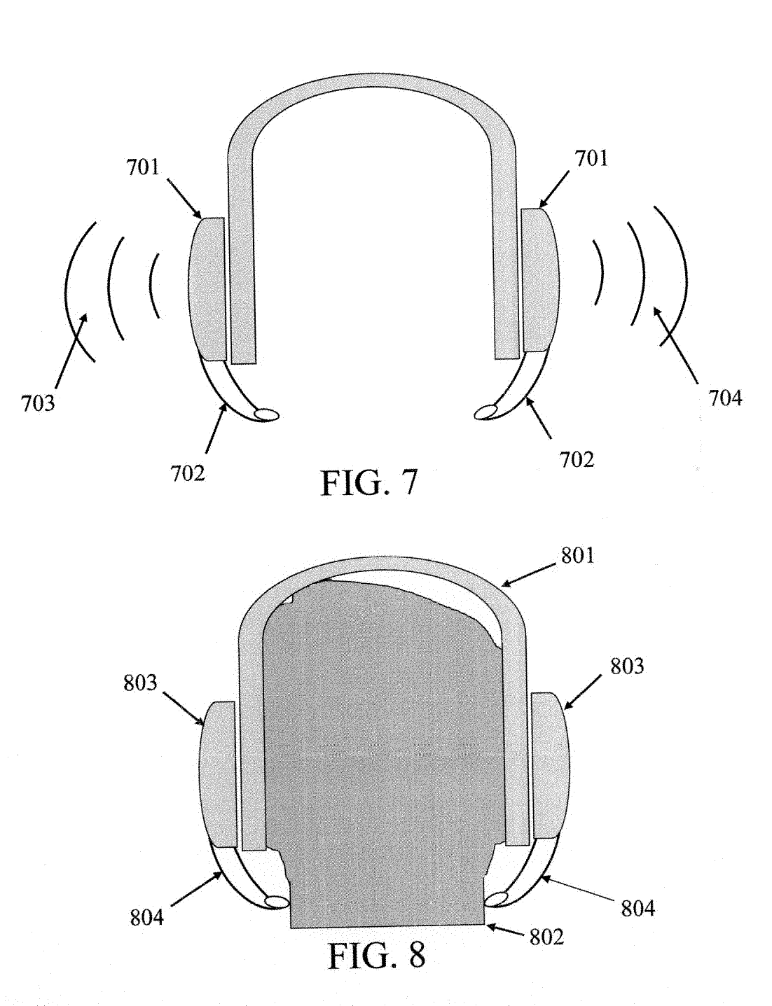

FIG. 7 is a perspective view of the disclosure configured for wireless inputs. Wireless signal 703 provides a means for control inputs and/or power inputs to headphone speakers 701. Wireless signal 704 provides a means for control inputs and/or power inputs to transdermal electrical contacts 702.

FIG. 8 illustrates the disclosure 801 as worn by user 802 in accordance with the preferred embodiment. Headphone speakers 803 provide auditory stimulation to a user's ears, while transdermal electrical contacts 804 provide somatosensory stimulation to a user's neck.

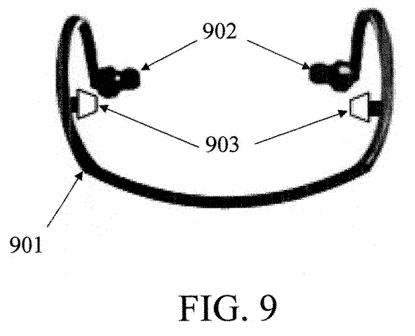

FIG. 9 is a perspective diagram of an embodiment of the present disclosure configured with a behind the neck type of headset. In this embodiment, housing 901 is worn behind a user's head (not shown) or neck (not shown). Speakers 902, which in this configuration are ear buds, provide auditory stimulation to a user's ears (not shown), while transdermal electrical contact/electrodes 903 provide somatosensory stimulation to a user's neck (not shown).

A method and devices for headphone with transdermal electrical nerve stimulation has been disclosed. The present disclosure anticipates numerous variations in the devices used. Numerous headphone or speaker designs can be used which can all provide the same function and have various features, including such features as being foldable, having on/off control, volume control, etc., which do not affect the function of the present disclosure. Likewise, numerous TENS designs can be used which can all provide the same function and have various features, including such features as on/off control, frequency control, waveform control, timing control, etc. The scope of the present disclosure should be construed broadly and is only to be limited to that which is claimed and all equivalents. Therefore, it is understood that the above description and illustrations are exemplary of the disclosure and are not to be considered as limiting; and the disclosure is understood to include any equivalents and is not considered as limited by the above description.

It is noted that the various modules, submodules, units, subunits, and components in the present disclosure can be implemented using any suitable technology. For example, a module or a unit can be implemented using processing circuitry. In an example, a module or a unit can be implemented using one or more integrated circuits (IC). In another example, a module or a unit can be implemented as one or more processors executing software instructions. In another example, interface circuitry is used to implement a receiving unit (or module) and/or a sending unit (or module).

What is claimed is:

1. An electronic wearable apparatus for providing bimodal stimulation to auditory system and somatosensory system of a user, comprising:
a head-mounted wearable housing attached to both auditory and somatosensory systems of the user;
a wearable audio output structure configured to apply audio stimulation to the auditory system of the user, the audio output structure having tunable frequency and signal tracking capability;
a wearable transdermal electrical transducer configured to apply a somatosensory stimulation signal to the somatosensory system of the user through a transdermal electrical contact which is in direct contact with a portion of surface structure of the user for stimulating the somatosensory system, and
an electronic control processor, being connected to the audio output structure and the transdermal electrical transducer, configured to synchronize the bimodal stimulations to create a nerve stimulation to improve user's body states by bimodal stimulation programming.

2. The apparatus of claim 1, wherein said audio output structure includes a speaker and transmits sound through a way of air-conducted or air-transmitted.

3. The apparatus of claim 1, wherein said audio output structure includes a bone conduction hearing structure and transmits sound through a way of bone-conducted or bone-transmitted.

4. The apparatus of claim 1, wherein said somatosensory stimulation signal passes from said transdermal electrical contact to the portion of the surface structure of the user through an electrode, the electrode being removable and replaceable.

5. The apparatus of claim 1 further comprising a spring tensioner to maintain positive contact between said transdermal electrical contact and said surface structure on a body part of the user.

6. The apparatus of claim 4, further comprising a spring tensioner to maintain a positive contact between said electrode and the portion of the surface structure of the user.

7. The apparatus of claim 1, wherein the electronic control processor includes at least one of a transdermal electrical nerve stimulation (TENS) control unit, an auditory control unit 102, or a somatosensory control unit.

8. The apparatus of claim 1, wherein said transdermal electrical contact is configured to be retracted or stored such that the apparatus can be used as a conventional headphone without somatosensory stimulation.

9. The apparatus of claim 1, further comprising a wire that transmits electrical impulses or other signals to power or control said audio output structure.

10. The apparatus of claim 1, further comprising a wire that transmits electrical impulses or other signals to power or control said transdermal electrical contact.

11. The apparatus of claim 1, further comprising circuitry that wirelessly transmits and receives electrical impulses or other signals to power or control said audio output structure.

12. The apparatus of claim 1, further comprising circuitry that wireless transmits and receives electrical impulses or other signals to power or control said transdermal electrical contact.

13. The apparatus of claim 1, further comprising an audio input device such as a microphone.

14. A method for providing bimodal stimulation which stimulates both auditory and somatosensory systems of a user, comprising:
providing the user a head-mounted wearable housing with electronic input and output;
providing the user a wearable audio output structure having tunable frequency and signal tracking capability that creates an audio stimulation for the user;
providing the user a wearable transdermal electrical transducer, the transducer being configured to apply a somatosensory stimulation signal to the somatosensory system of the user through a transdermal electrical contact that is in direct contact with a portion of a surface structure of the user for stimulating the somatosensory system, and
having an electronic control processor to control the audio output structure and the transdermal electrical transducer, and synchronize the bimodal stimulations to create a nerve stimulation to improve user's body states.

15. The method of claim 14, further comprising a spring tensioner to maintain positive contact between said transdermal electrical contact and said portion of the surface structure of the user.

16. An apparatus for providing bimodal stimulation to an auditory system and a somatosensory system of a user, comprising:
a housing;
an audio output component configured to create an audio stimulation for the user; and
a transdermal electrical component configured to apply a somatosensory stimulation signal to the somatosensory system of the user through a transdermal electrical contact that is in direct contact with a portion of a surface structure the user for stimulating the somatosensory system.

17. The apparatus of claim 16, wherein said somatosensory stimulation signal passes from said transdermal electrical contact to the portion of the surface structure of the user through an electrode.

18. The apparatus of claim 16, further comprising a spring tensioner to maintain positive contact between said transdermal electrical contact and said portion of the surface structure of the user.

19. The apparatus of claim 16, further includes a battery that is configured to storage of electricity.

20. The apparatus of claim 19, wherein the battery is rechargeable.
